(19) United States
(12) Patent Application Publication
(10) Pub. No.: US 2026/0166317 A1
(43) Pub. Date: Jun. 18, 2026

(54) STIMULATION SYSTEMS AND METHODS THEREFOR

(71) Applicant: Lungpacer Medical Inc., Vancouver (CA)

(72) Inventors: Thiago BASSI, Burlington (CA); Douglas EVANS, Downingtown, PA (US)

(73) Assignee: Lungpacer Medical Inc., Vancouver, BC (CA)

(21) Appl. No.: 19/128,538

(22) PCT Filed: Nov. 10, 2023
(86) PCT No.: PCT/IB2023/061394

§ 371 Date: May 8, 2025

Related U.S. Application Data
(60) Provisional application No. 63/383,346, filed on Nov. 11, 2022.

(51) Int. Cl. A61N 1/36; A61N 1/04; A61N 1/05

(52) U.S. Cl. CPC A61N 1/3603; A61N 1/0456; A61N 1/0492; A61N 1/0546; A61N 1/0548; A61N 1/36025; A61N 1/36036

(57) ABSTRACT

Methods of stimulation may include delivering stimulation to a pterygopalatine ganglia, a celiac ganglion, a superior cervical ganglia, a great petrosal nerve, a posterior superior alveolar nerve, or a combination thereof. Delivering the stimulation may modulate an activity of a brain network, improve flow of a glymphatic system, and/or modulate a myogenic activity in cerebral blood vessels. Modulating the activity of the brain network may include modulating the electrical activity of one or more regions of the brain and/or modulating the release of one or more neurotransmitters.

### CROSS-REFERENCE TO RELATED APPLICATIONS

This application claims priority to U.S. Provisional Application No. 63/383,346, filed on Nov. 11, 2022, which is hereby incorporated by reference in its entirety.

In general, all publications and patent applications mentioned in this specification are herein incorporated by reference in their entirety to the same extent as if each individual publication or patent application was specifically indicated to be incorporated by reference. For example, embodiments of the present disclosure may be used in combination with one or more systems, catheters, stimulators, apparatuses, and electrodes described in U.S. Pat. Nos. 9,242,088, 9,333,363, 9,776,005, 10,039,920, 10,293,164, 10,940,308, 10,987,511, 11,357,979, U.S. Pat. Pub. No. 2019/0001126, U.S. Pat. Pub. No. 2020/0391027, U.S. Pat. Pub. No. 2022/0134095, and/or U.S. Pat. Pub. No. 2023/0023475; the disclosures of which are hereby incorporated by reference.

### INTRODUCTION

The embodiments of this disclosure generally relate to methods and devices (including systems) for the stimulation of nerves, muscles, and/or other body tissue. More specifically, embodiments of the present disclosure include methods and systems for modulating one or more brain networks, improving glymphatic system flow, and promoting cerebral autoregulation.

### SUMMARY

Embodiments of the present disclosure relate to, among other things, systems, devices, and methods for modulating an activity of a brain network, improving flow of a glymphatic system, and/or modulating a myogenic activity in cerebral blood vessels. Embodiments include systems and devices for applying stimulation to one or more anatomical targets. In some embodiments, the stimulation applied to one or more anatomical targets may be adjusted based on a measured physiological parameter. Each of the embodiments disclosed herein may include one or more of the features described in connection with any of the other disclosed embodiments.

In one example, a method of stimulation comprises delivering stimulation to a pterygopalatine ganglia, a celiac ganglion, a superior cervical ganglia, a great petrosal nerve, a posterior superior alveolar nerve, or a combination thereof. The stimulation may modulate an activity of a brain network, improve flow of a glymphatic system, and/or modulate a myogenic activity in cerebral blood vessels. The stimulation may activate smooth muscle cells in a cerebral blood vessel. The brain network may include a default mode network, a salience network, a dorsal attention network, and/or a frontal-parietal network. Modulating the activity of the brain network may include modulating the electrical activity of one or more regions of the brain associated with the brain network, such as, for example, a thalamus, a hippocampus, and/or a prefrontal cortex. In addition or alternatively, modulating the activity of the brain network may include increasing the production of dopamine, acetylcholine, n-methyl-D-aspartate, gamma-aminobutyric acid, cerebral adenosine triphosphate, cerebral calcium, and/or noradrenaline.

### BRIEF DESCRIPTION OF THE DRAWINGS

The accompanying drawings, which are incorporated in and constitute part of this specification, illustrate non-limiting embodiments of the present disclosure and together with the description, serve to explain the principles of the disclosure.

FIG. 1A illustrates a perspective view of exemplary oral stimulator, according to one or more embodiments;

FIG. 1B illustrates a top view of the oral stimulator of FIG. 1A;

FIG. 2 illustrates an exemplary aural stimulator, according to one or more embodiments;

FIG. 3A illustrates an exemplary nasal stimulator, according to one or more embodiments;

FIG. 3B illustrates the nasal stimulator of FIG. 3A, rotated 90° about a longitudinal axis;

FIG. 4 illustrates a graphical representation of data transmitted from one or more sensors to a control unit, according to one or more embodiments;

FIG. 5 illustrates an exemplary stimulation system, according to one or more embodiments;

FIG. 6 illustrates an exemplary stimulation system, according to one or more embodiments; and

FIG. 7 illustrates an exemplary stimulation system, according to one or more embodiments.

### DETAILED DESCRIPTION

Embodiments of the present disclosure include methods and stimulation systems. Methods and systems of the present disclosure may be configured to stimulate one or more anatomical targets, such as for example, one or more phrenic nerves, one or more vagus nerves (e.g., a cervical portion, a thoracic portion, and/or an inner ear portion), a maxillary nerve, a pterygopalatine ganglia, a celiac ganglion, a superior cervical ganglia, a greater petrosal nerve, and/or a posterior superior alveolar nerve. The stimulation of one or more anatomical targets may modulate one or more brain networks (e.g., a default mode network, a salience network, a dorsal attention network, and/or a frontal-parietal network), promote cerebral autoregulation and/or improve glymphatic system drainage. As used herein, modulating a brain network may refer to changing, adjusting, altering, increasing, or decreasing, chemical and/or electrical activity of the brain network. For example, embodiments that modulate one or more brain networks may modulate electrical activity in a thalamus, a hippocampus, a limbic structure, a paralimbic structure, and/or a prefrontal cortex. In addition or alternatively, embodiments that modulate one or more brain networks may modulate the release of one or more neurotransmitters.

The modulation one or more brain networks, promotion of cerebral autoregulation, and/or improvement of glymphatic system drainage, may result in better outcomes in critical and non-critical patients.

The respiratory system of a subject is responsible for the exchange of carbon dioxide and oxygen between the subject and the atmosphere. This exchange may be referred to as the respiratory cycle. Characteristics of the respiratory cycle (e.g., rate, volume, and/or quality of breaths taken by the subject) affect the relative levels of carbon dioxide and oxygen within the subject. The respiratory cycle affects the respiratory system, the cardiovascular system, other tissues, and can affect physiological, behavioral, and cognitive processes.

For example, the frequency and the rate of breathing may assist in modulating mood disorders and increasing attention and focus. Breathing techniques may assist in managing panic attacks, anxiety, and depression. Breathing exercises may also modulate brain activity, thereby reducing stress and burnout syndrome.

Nasal breathing and diaphragmatic breathing techniques may modulate brain activity, thereby reducing damage to the brain. For example, breathing without components of nasal breathing and diaphragmatic breathing may adversely affect the brain. Nasal and diaphragmatic breathing may generate hippocampal activity. The generated hippocampal activity coupled with nasal and diaphragmatic breathing may reduce hippocampal neuroinflammation.

External respiratory support, such as, for example, mechanical ventilation, may be indicated for critically ill patients. However, mechanical ventilation is associated with changes in neural activity. For example, mechanical ventilation may be associated with default mode brain network. A brain network is a collection of widespread brain regions showing functional connectivity. The default mode network includes regions of the medial prefrontal cortex, hippocampal formation, and the posterior cingulate gyrus.

The default mode network may have increased activity during certain activities, such as, for example, daydreaming, recalling memories, envisioning the future, monitoring the environment, or thinking about the intentions of others. Unbalanced or atypical activity in the default mode network may correlate with mental disorders, including depression, anxiety, delirium, and schizophrenia. For example, activity in the posterior cingulate gyrus may be increased in subjects experiencing delirium, and activity in the posterior cingulate gyrus may be reduced when delirium symptoms cease.

Therapies including breathing exercises (e.g., meditation) may influence activity in the default mode network, and improve patient outcomes. Activity in the posterior cingulate gyrus is positively correlated with the tidal volume delivered by mechanical ventilation. Subjects undergoing ventilation at a tidal volume of less than or equal to approximately 2 ml/kg may exhibit lower neuronal activity compared to subjects undergoing ventilation at a tidal volume of approximately 30 ml/kg.

Subjects undergoing mechanical ventilation may show a reduction in gamma wave oscillations. Delivery of air to the nasal passages of the subject (e.g., an air puff) in synchrony with mechanical ventilation can increase gamma wave oscillations, compared to mechanical ventilation alone.

Physiological changes in a subject may result in signals being transmitted to the brain stem, from the one or more phrenic nerves, one or more vagus nerves, or a combination thereof. For example, physiological changes in the body resulting from variations in the respiratory cycle, respiratory muscles tonus, serum pH, pulmonary tissue stretch, oxygen serum concentration, and/or arterial baroreceptors activity, may result in signals being transmitted to the brain stem from one or more phrenic and/or vagus nerves.

Information received from one or more phrenic and/or vagus nerves may assist the respiratory center of the brainstem in modulating respiration and cognitive function.

The modulation of cognitive functions by respiration includes signals transferred via a constant connection between the brainstem and prefrontal cortex, especially the medial prefrontal cortex and supplementary premotor cortex. Physiological feedback between the respiratory center located in the brainstem and supratentorial areas assist in the regulation of dopamine levels within a normal physiological range. Interruption of the physiological feedback between the respiratory center located in the brainstem and supratentorial areas can result in an unregulated or abnormal release of neurotransmitters in the prefrontal cortex. Respiration techniques, or other regulation of the respiratory cycle, can reestablish the physiological feedback between the respiratory center located in the brainstem and supratentorial areas, improving outcomes for subjects experiencing panic or anxiety attacks.

Negative pressure is generated in a subject during normal respiratory activity, by, for example, a diaphragm and/or accessory respiratory muscles. The negative pressure generated during normal respiratory activity can assist in regulating the release of neurotransmitters in the prefrontal cortex. The negative pressure also promotes brain venous return and glymphatic system drainage. Promotion of glymphatic system drainage may promote activity of the glymphatic system, which assists in cleaning toxic proteins, inflammatory debris, and products of brain cell degeneration.

Additionally, negative pressure generated by the diaphragm and/or accessory respiratory muscles assists cerebral autoregulation. Cerebral autoregulation refers to the subject's ability to adjust the diameter of the cerebral vessels in order to keep cerebral flow constant during variations in arterial blood pressure. During mechanical ventilation, communication between the respiratory center and the prefrontal cortex changes due to sedation, impaired neuronal synapsis, reduction in the diaphragmatic tonus, and the absence of negative pressure. These factors trigger neuroinflammation and may impair cerebral autoregulation. Therefore, the diaphragmatic tonus, the negative pressure generated by the diaphragm, and the physiological feedback signals coming from the spinal cord, phrenic nerves, and vagus nerves assist in the regulation of prefrontal cortex activity by keeping the release of neurotransmitters in a physiological range, and assisting cerebral autoregulation.

Cerebral autoregulation is dependent on the communication between the nerves and the cerebral blood vessels. Two main pathways connect nerves to the cerebral vessels. One pathway, the direct or central pathway, includes the modulation of cerebral blood vessel diameter via multi-synapse neurons in the brainstem (e.g., the locus coeruleus, part of the sympathetic system), to keep cerebral brain perfusion constant. The second pathway, the indirect or peripheral pathway, includes the modulation of the cerebral pressure perfusion via the activity of the optic ganglia and the pterygopalatine ganglia. Both the optic ganglia and the pterygopalatine ganglia are directly connected to the nucleus of the tractus solitarius in the brainstem (part of the parasympathetic system).

The parasympathetic system innervates the cerebral vessels through three main anatomical ganglia, the cervical superior ganglia, the optic ganglia, and the pterygopalatine ganglia. The parasympathetic system is comprised of several nerves, including the trigeminus, facial, glossopharyngeal, and vagus nerves. The facial nerve is a cranial nerve that is sensory and motor. The sensory part of the facial nerve may also be referred to as the intermediate nerve. The intermediate nerve leaves the brainstem traveling through the internal acoustic meatus (e.g., the petrous part of the temporal bone). Within the petrous bone, the intermediate nerve is proximate to the inner ear. Within the temporal bone, the nerve root of the intermediate nerve enters the facial canal, where the intermediate nerve and the motor root of the facial nerve meet, forming the facial nerve. The nerve roots of the intermediate nerve come together to form the geniculate ganglion. Three nerves also branch from where the nerve root of the intermediate nerve enters the facial canal, the greater petrosal nerve (exclusively with parasympathetic fibers), the nerve to stapedius (exclusively motor fibers to stapedius muscle), and chorda tympani (special sensory fibers to the anterior two-thirds of the tongue and parasympathetic fiber to the submandibular and sublingual glands). The parasympathetic fibers of the facial nerve are carried by the greater petrosal nerve and chorda tympani branches. The greater petrosal nerve arises immediately distal to the geniculate ganglion within the facial canal.

On the opposite side of the temporal bone from the inner ear, the greater petrosal nerve combines with the deep petrosal nerve to form the nerve of the pterygoid canal, entering the pterygopalatine fossa and connecting with the pterygopalatine ganglion. The nerve of the pterygoid canal provides parasympathetic innervation to the mucous gland of the oral cavity, nose, pharynx, lacrimal gland, and cerebral brain vessels. The superior cervical ganglia are formed by the junction of sympathetic fibers coming from and between the C5 to T1 vertebrae. Between the superior cervical ganglia and the cerebral blood vessels, the sympathetic fibers are proximate to the carotid artery.

Embodiments of the present disclosure may include methods and systems for modulating brainstem activity, diaphragm tonus, and/or respiratory cycle variations. Modulating brainstem activity, diaphragm tonus, and/or respiratory cycle variations of a subject may modulate the respiratory drive of the subject. Methods and systems for delivering stimulation described herein may allow for the modulation of the autonomic system of a subject, including modulating the balance of the subject's autonomic system towards either parasympathetic or sympathetic activity.

Embodiments of the present disclosure include methods and systems for modulating brain network activity and cerebral autoregulation via peripheral nerve stimulation. For example, embodiments of the present disclosure may include modulation of the default mode network and/or other brain networks. Embodiments may include direct stimulation of one or more phrenic nerves. Stimulation of one or more phrenic nerves may result in a retrograde phrenic nerve signal to the phrenic nucleus within the spinal cord, which results in a plurality of signals being transmitted via multiple interneuron connections to one or more respiratory centers in the brainstem, such as, for example, nucleus of tractus solitarius and/or locus coeruleus. The retrograde stimulation of one or more respiratory centers in the brainstem modulates the activity in the thalamus, hippocampus, limbic and paralimbic structures, prefrontal cortex, and one or more brain networks. For example, retrograde stimulation of one or more respiratory centers in the brainstem may increase hippocampus neurogenesis, increase hippocampus astrogenesis, decrease hippocampus inflammation, increase lung compliance, increase prefrontal cortex activity, increase brain stem activity, and increase trigeminal nerve activity. The retrograde stimulation of one or more respiratory centers in the brain may also modulate cerebral autoregulation.

Embodiments of the present disclosure may modulate the brain network's activity and cerebral autoregulation by directly stimulating the pterygopalatine ganglia. Some embodiments may also modulate the activity of the brain networks by indirectly stimulating the celiac ganglion via direct stimulation of the left phrenic nerve as well as direct stimulation of the superior cervical ganglia.

Methods of modulating brain network activity may include the stimulation of a peripheral nerve during mechanical ventilation. The peripheral nerve may include a phrenic nerve, a vagus nerve, and/or a celiac ganglion. The modulation of brain network activity may include changes in brain wave oscillations and neuronal activity (e.g., neuronal activity in thalamus, hippocampus, limbic and paralimbic structures, and/or prefrontal cortex). Modulation of brain network activity may include modulation of the default mode network, the salience network, the dorsal attention network, the frontal-parietal network, and/or cerebral autoregulation.

For example, modulation of brain network activity may be achieved by the modulation of the release of neurotransmitters, in addition to the modulation of the nerve signalling from the peripheral nerves reaching the brain. For example, stimulation of a peripheral nerve during mechanical ventilation may modulate the nerve signalling from the peripheral nerves to the brain, thereby modulating the release of neurotransmitters. Modulating the release of neurotransmitters may include increasing the production of dopamine, acetylcholine, n-methyl-D-aspartate (NMDA), gamma-aminobutyric acid (GAMA), cerebral adenosine triphosphate (ATP), cerebral calcium, and/or noradrenaline.

Embodiments of the present disclosure may improve outflow in the glymphatic system, improve brain venous return, and/or improve cerebral autoregulation, thereby assisting in the cleaning and removal of inflammatory debris and toxic proteins. For example, stimulation may be delivered to a peripheral nerve, thereby improving outflow of the glymphatic system, brain venous return, and/or cerebral autoregulation.

Embodiments of the present disclosure may include stimulation of a vagus nerve (e.g., cervical, thoracic, and/or inner ear portions of a vagus nerve). Some embodiments may modulate the activity of the brain network by stimulating the vagus nerve, thereby increasing the pulmonary afferent signal from the parasympathetic receptors and increasing vagal activity. One or more embodiments may also modulate the activity of the brain networks and cerebral autoregulation by indirectly modulating the afferent signal from the respiratory system to the central nerve system. Embodiments of the present disclosure may include direct stimulation of the parasympathetic system, such as, for example, stimulation of the pterygopalatine ganglia and/or the greater petrosal nerve. In addition or alternatively, embodiments of the present disclosure may include direction stimulation (e.g., using magnetic and/or electrical energy) of a prefrontal cortex, a supplementary motor cortex, a diaphragm motor cortex, or a combination thereof.

Stimulation of pterygopalatine ganglia, a greater petrosal nerve, and/or a peripheral nerve may activate smooth muscle in the arterioles, thereby modulating cerebral autoregulation. Delivery of stimulation to cerebral vessels may assist in modulating cerebral autoregulation.

The present disclosure describes stimulation systems configured to provide one or more channels of stimulation, as described herein. A stimulation system may include an oral stimulator, an aural stimulator, a nasal stimulator, an oral endoscope, a transvascular catheter, ear clip, an electromagnetic field generator, cranial stimulator, and/or one or more dermal patches. Each of the oral stimulator, aural stimulator, nasal stimulator, oral endoscope, transvascular catheter, ear clip, electromagnetic field generator, cranial stimulator and/or dermal patches may be controlled separately by a control unit of the stimulation system. The oral stimulator, aural stimulator, nasal stimulator, oral endoscope, transvascular catheter, ear clip, electromagnetic field generator, cranial stimulator and dermal patches may include one or more stimulations channels, each.

The stimulation system may be connected to one or more energy sources, including for example, an electrical source. Electrical sources may include alternating current (e.g., wall power), direct current (e.g., battery power), or other sources of electrical energy. One or more components of the stimulation system may be configured to deliver electrical energy (discussed further below) at low frequencies (e.g., approximately 1 Hertz (Hz) to approximately 45 Hz) and/or high frequencies (e.g., approximately 10,000 Hz). The electrical energy may be delivered in pulses, where each pulse independently has a width of approximately 10 milliseconds (ms) to approximately 500 ms, and the total duration of each delivery of electrical stimulation is approximately 0.1 seconds to approximately 3.0 seconds (e.g., approximately 0.1 seconds to 2.0 seconds during inspiration and/or approximately 0.1 seconds to 3.0 seconds during expiration). The amplitude of delivered electrical energy may vary from approximately 1 milliampere (mA) to approximately 20 mA. In addition or alternatively, one or more components of the system may be configured to deliver magnetic stimulation, infrared stimulation, and/or thermal stimulation.

As described herein, physiological data related to a subject's response to stimulation may be monitored, tracked, and/or recorded. Stimulation parameters of the delivered stimulation may be modified based on the subject's response to stimulation. For example, if low frequency stimulation is ineffective at regulating one or more brain networks, the stimulation system may increase the frequency and/or amplitude of stimulation delivered. The adjustment of stimulation parameters based on a subject's physiological response to stimulation may allow for the stimulation system to modulate the balance of the subject's autonomic system towards either parasympathetic or sympathetic activity, in order to cause modulation of one or more brain networks.

In some embodiments, the stimulation delivered from multiple channels includes the same stimulation parameters (e.g., amplitude, charge, frequency, pulse width, duration of stimulation). Stimulation system may be configured to deliver stimulation from multiple channels with different stimulation parameters. For example, the stimulation delivered via oral stimulator and/or nasal stimulator may include different stimulation parameters than the stimulation delivered via a transvenous catheter.

### Oral Stimulator

A stimulation system may include an oral stimulator. The oral stimulator may be configured to deliver electrical stimulation to one or more anatomical targets proximate a mouth of the subject. The oral stimulator may include one or more sensors configured to receive data corresponding to nerve activity of a maxillary nerve, a pterygopalatine ganglia, and/or a posterior superior alveolar nerve. In addition or alternatively, the oral stimulator may include one or more sensors configured to receive data corresponding to a pH and/or a temperature of the subject's mouth.

The oral stimulator may include one or more electrodes. In some embodiments, each of the one or more electrodes may be configured to receive signals (e.g., act as a sensor receiving data corresponding to a nerve activity) and deliver signals (e.g., stimulation to one or more anatomical targets of the subject.) For example, when in place within the mouth of a subject, one or more electrodes of the oral stimulator may be positioned to deliver stimulation to a maxillary nerve, a pterygopalatine ganglia, a posterior superior alveolar nerve, an efferent nerve fiber of the posterior superior alveolar nerve, and/or an afferent nerve fiber of the superior alveolar nerve. In addition or alternatively, one or more electrodes of the oral stimulator may be positioned to receive electrical impulses indicative of a nerve activity of a maxillary nerve, a pterygopalatine ganglia, and/or a posterior superior alveolar nerve.

The oral stimulator may be in wireless communication with the control unit of the stimulation system. The oral stimulator may transmit received data to the control unit. The control unit may send instructions to the oral stimulator, including stimulation parameters.

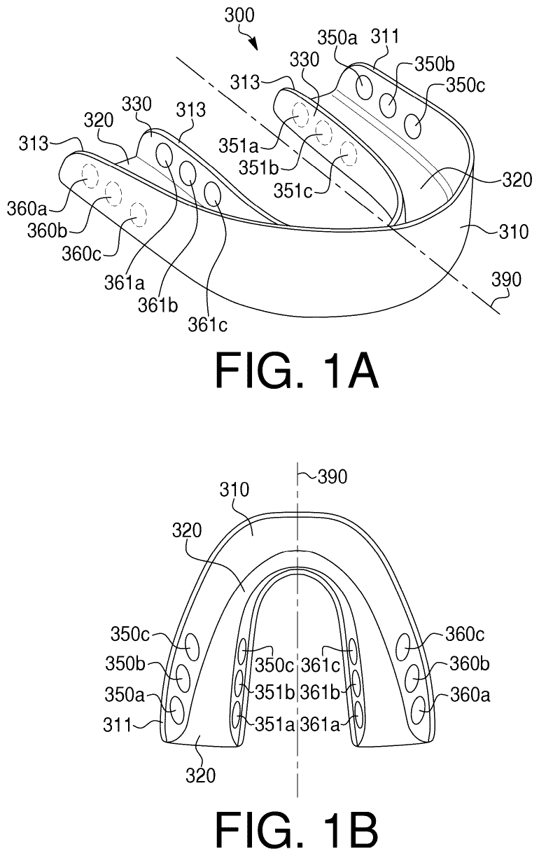

As described herein, a stimulation system may include an oral stimulator 300. One exemplary configuration of an oral stimulator 300 is shown in FIGS. 1A and 1B. The oral stimulator 300 shown in FIG. 1B is the same oral stimulator shown in FIG. 1A, but FIG. 1A provides a perspective view, and FIG. 1B provides a top view.

Oral stimulator 300 may include a front wall 310, a bottom wall 320, and/or a rear wall 330. Bottom wall 320 may join front wall 310 to rear wall 330. Front wall 310 may extend from bottom wall 320 to a top edge 311 of front wall 310. Rear wall 330 may extend from bottom wall 200 to a top edge 313 of rear wall 330.

Front wall 310 may have a height of approximately 1 millimeter (mm) to approximately 10 mm. Rear wall 330 may have a height of approximately 1 mm to approximately 10 mm. Oral stimulator 300 may be configured such that a distance between front wall 310 and rear wall 330 is approximately 7 centimeters (cm) to approximately 10 cm.

Oral stimulator 300 may comprise a flexible material. For example, oral stimulator 300 may comprise a material with a flexibility such that the oral stimulator 300 conforms to an anatomy of the patient. In some embodiments, oral stimulator 300 may comprise silicone, a rubber (e.g., a latex rubber), a plastic (e.g., a polyethylene-polyvinylacetate copolymer (EVA), polyvinyl chloride, a polyether block amide), stainless steel, or a combination thereof.

When oral stimulator 300 is positioned for use (e.g., within the mouth of a subject), the subject's upper teeth may rest between the front wall 310 and the rear wall 330. The front wall 310 may extend between the upper lip of the subject and the upper teeth of the subject. The rear wall 330 may extend between the upper teeth of the subject and a tongue of the subject. Top edge of rear wall 313 may contact the hard palate. The biting surface of the upper teeth of the subject may contact bottom wall 320.

The exemplary oral stimulator 300 shown in FIGS. 1A and 1B is symmetrical. A medial axis 390, along the plane of symmetry of the oral stimulator 300, is shown in FIGS. 1A and 1B. In other embodiments, oral stimulator 300 may be asymmetrical. For example, oral stimulator 300 may include electrodes (e.g., left lateral electrodes 350, left medial electrodes 351, right lateral electrodes 360, right medial electrodes 361), on only one side of medial axis 390 (e.g., only left electrodes or only right electrodes). In some embodiments, the shape of oral stimulator 300, including walls 310, 320, and 330, may be configured to conform to a subject's anatomy (e.g., shape, size, and location of teeth, palate, and/or gums).

Oral stimulator 300 may include one or more electrodes configured to align with roots of one or more molars of the subject. For example, oral stimulator 300 may include one or more left lateral electrodes 350 (e.g., 350a, 350b, and 350c) positioned on a surface of front wall 310 and/or one or more left medial electrodes 351 (e.g., 351a, 351b, and 351c) positioned on a surface of rear wall 330. The left lateral electrodes 350 may be configured such that, when the oral stimulator 300 is in position for use (e.g., within the mouth of the subject), the left lateral electrodes 350 align with nerve tissue above one or more teeth of a subject. The left medial electrodes 351 may be configured such that, when the oral stimulator 300 is in position for use (e.g., within the mouth of the subject), the left medial electrodes 351 align with nerve tissue above one or more teeth of a subject. In some embodiments, left medial electrodes 351 are positioned along rear wall 330 at locations across from corresponding left lateral electrodes 350. When oral stimulator 300 is in use, stimulation (e.g., electrical signals) may flow bi-directionally between a left medial electrode 351 and the corresponding left lateral electrode 350 (e.g., between electrode 351a and electrode 350a), and through an anatomical target (e.g., a left posterior superior alveolar nerve, an afferent nerve fiber of a left posterior superior alveolar nerve, an efferent nerve fiber of a left posterior superior alveolar nerve, a maxillary nerve, and/or a pterygopalatine ganglia).

Oral stimulator 300 may include one or more right lateral electrodes 360 (e.g., 360a, 360b, and 360c) positioned on a surface of front wall 310 and/or one or more right medial electrodes 361 (e.g., 360a, 360b, and 360c) positioned on a surface of rear wall 330. The right lateral electrodes 360 may be configured such that, when the oral stimulator 300 is in position for use (e.g., within the mouth of the subject), the right lateral electrodes 360 align with nerve tissue above one or more teeth of a subject. The right medial electrodes 361 may be configured such that, when the oral stimulator 300 is in position for use (e.g., within the mouth of the subject), the right medial electrodes 361 align with nerve tissue above one or more teeth of a subject. In some embodiments, right medial electrodes 361 are positioned along rear wall 330 at locations across from corresponding right lateral electrodes 360. When oral stimulator 300 is in use, stimulation (e.g., electrical signals) may flow bi-directionally between a right medial electrode 361 and the corresponding right lateral electrode 360 (e.g., between electrode 361a and electrode 360a), and through an anatomical target (e.g., a right posterior superior alveolar nerve, an afferent nerve fiber of a right posterior superior alveolar nerve, an efferent nerve fiber of a right posterior superior alveolar nerve, a maxillary nerve, and/or a pterygopalatine ganglia). Anatomical targets of oral stimulator 300 may include tissue above one or more upper teeth and/or below one or more lower teeth.

In some embodiments, a set of electrodes, such as, for example, left lateral electrodes 350, left medial electrodes 351, right lateral electrodes 360, or right medial electrodes 361, may be aligned along an axis. In other embodiments, electrodes within a set may be located a different vertical positions along front wall 310 or rear wall 330. Each set of electrodes, such as, for example, left lateral electrodes 350, left medial electrodes 351, right lateral electrodes 360, or right medial electrodes 361, may include one or more electrodes (e.g., 1 electrode per set, 2 electrodes per set, 3 electrodes per set, four electrodes per set, five electrodes per set). In some embodiments, each set of electrodes may have the same number of electrodes. Alternatively, some sets of electrodes may include more electrodes than other sets of electrodes.

Referring to FIGS. 1A and 1B, oral stimulator 300 may include a first left lateral electrode 350a and a first left medial electrode 351a positioned such that, when the oral stimulator 300 is in position for use (e.g., within the mouth of the subject), a left first molar of the subject is between the first left lateral electrode 350a and the first left medial electrode 351a. Oral stimulator 300 may include a second left lateral electrode 350b and a second left medial electrode 351b positioned such that, when the oral stimulator 300 is in position for use (e.g., within the mouth of the subject), a left second molar of the subject is between the second left lateral electrode 350b and the second left medial electrode 351b. Oral stimulator 300 may include a third left lateral electrode 350c and a third left medial electrode 351c positioned such that, when the oral stimulator 300 is in position for use (e.g., within the mouth of the subject), a left third molar of the subject is between the third left lateral electrode 350c and the third left medial electrode 351c.

Oral stimulator 300 may include a first right lateral electrode 360a and a first right medial electrode 361a positioned such that, when the oral stimulator 300 is in position for use (e.g., within the mouth of the subject), a right first molar of the subject is between the first right lateral electrode 360a and the first right medial electrode 361a. Oral stimulator 300 may include a second right lateral electrode 360b and a second right medial electrode 361b positioned such that, when the oral stimulator 300 is in position for use (e.g., within the mouth of the subject), a right second molar of the subject is between the second right lateral electrode 360b and the second right medial electrode 361b. Oral stimulator 300 may include a third right lateral electrode 360c and a third right medial electrode 361c positioned such that, when the oral stimulator 300 is in position for use (e.g., within the mouth of the subject), a right third molar of the subject is between the third right lateral electrode 360c and the third right medial electrode 361c.

### Aural Stimulator

A stimulation system may include an aural stimulator. The aural stimulator may be configured to be placed within a subject's ear, such as, for example, within an ear canal of the subject. The aural stimulator may be configured to deliver electrical stimulation to one or more anatomical targets proximate an ear canal of the subject. The aural stimulator may include a sensor configured to collect data related to the pressure being applied to the aural stimulator (e.g., from walls of the ear canal). The aural stimulator may also include a sensor configured to measure a distance between the cranial and ventral walls of the ear canal. Measurements of the pressure applied from the walls of the ear canal, and/or the distance between cranial and ventral walls of the ear canal, may be used to determine an intracranial pressure of the subject.

In some embodiments, aural stimulator includes a sensor configured to emit and receive sub-audible waves. The data regarding the transmitted and received sub-audible waves may be processed (e.g., by the aural stimulator or a connected control unit) to determine a tension in a membrane of tympanums of the subject. The tension of the membrane of tympanums correlates to parasympathetic nerve activity. One or more sensors of the aural stimulator may be configured to receive data corresponding to a temperature and/or pH of the ear canal.

The aural stimulator may include one or more electrodes. In some embodiments, each of the one or more electrodes may be configured to receive signals (e.g., act as a sensor receiving data corresponding to a pressure, a tension, a temperature, and/or pH) and deliver signals (e.g., stimulation to one or more anatomical targets of the subject). For example, when in place within the ear canal of the subject, one or more electrodes of the aural stimulator may be positioned to deliver stimulation to a maxillary nerve, a pterygopalatine ganglia, a superior cervical ganglia, and/or a great petrosal nerve.

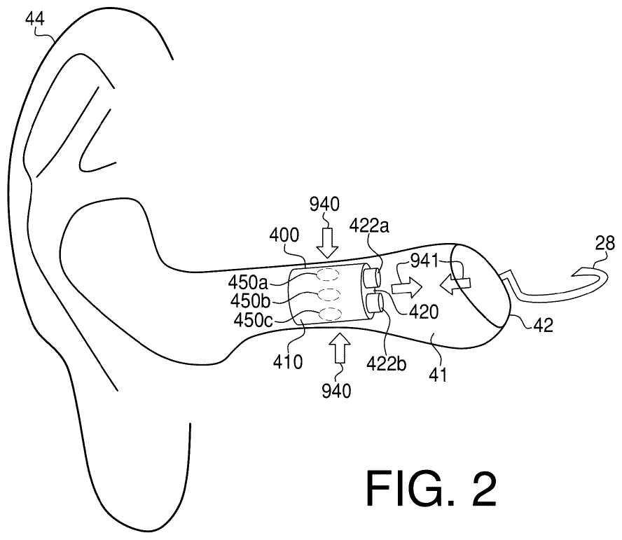

One exemplary configuration of an aural stimulator 400 is shown in FIG. 2. The aural stimulator 400 may be configured to be placed in the ear canal 41 of a subject. For example, aural stimulator 400 may be placed between an outer ear 44 and a membrane of tympanums 42 of the subject. Aural stimulator 400 may have a width (e.g., a diameter) of approximately 5 mm to approximately 20 mm.

Aural stimulator 400 may include a body including a medial surface 410, a proximal surface 420, and a distal surface (not shown). For example, aural stimulator 400 may have a cylindrical shape, where the proximal surface 420 is circular and parallel to a circular distal surface. The medial surface 410 may be a curved surface that connects the proximal surface 420 to the distal surface. When in position for use (e.g., within an ear canal of the subject), the proximal surface 420 may be closer to a membrane of tympanums 42 of the subject, than the distal surface.

Aural stimulator 400 may include one or more aural electrodes 450. For example, aural stimulator 400 may include one or more aural electrodes 450 positioned along at least a portion of a circumference of medial surface 410. Although only three aural electrodes 450a, 450b, 450c are shown in FIG. 2, electrodes may positioned around the entire circumference of medial surface 410. In some embodiments, aural stimulator 400 may include one or more infrasonic sensors 422. Infrasonic sensors 422a, 422b may be configured to emit and receive sound waves 941 at a frequency below the human range of hearing. For example, infrasonic sensors 422a, 422b may emit and receive sound waves at frequencies of approximately 0.1 Hz to approximately 20.0 Hz. Emitted sound waves 941 may be directed to a membrane of tympanums 42 of the subject, the sound waves 941 may be reflected off the membrane of tympanums 42, and received by infrasonic sensors 422a, 422b.

As shown in FIG. 2, the aural stimulator 400 may be placed within an ear canal of a subject. One or more sensors (e.g., aural electrodes 450) may measure the pressure 940 being applied on aural stimulator 400 by walls of the ear canal 41. Depending on physiological characteristics of the subject, the pressure 940 applied on aural stimulator 400 by walls of the ear canal 41 may vary. For example, increases in intracranial pressure may cause an increase in pressure 940 applied on the aural stimulator 400, and decreases in intracranial pressure may cause a decrease in pressure 940 applied on the aural stimulator 400.

Aural stimulator 400 may comprise a material with a flexibility such that aural stimulator 400 conforms to an anatomy of the subject. For example, aural stimulator 400 may comprise silicone, a rubber (e.g., a latex rubber), a plastic (e.g., a polyethylene-polyvinylacetate copolymer (EVA), polyvinyl chloride, a polyether block amide), stainless steel, or a combination thereof. In addition or alternatively, aural stimulator 400 may comprise one or more compressible foams (e.g., a polyethylene or a polyamide). The compressible foam may be capable of being deformed for entry into an ear canal of a subject, then re-expand aural stimulator 400 is in place.

### Nasal Stimulator

A stimulation system may include a nasal stimulator. The nasal stimulator may be configured to deliver gas and/or electrical stimulation to one or more anatomical targets proximate a nasal cavity of the subject. For example, gas delivered to a subject via the nasal stimulator may stimulate one or more nerve fibers. The one or more nerve fibers may include an olfactory bulb, a pterygopalatine ganglia, and/or a pharyngeal branch of a vagus nerve. In addition or alternatively, gas delivered to the subject via the nasal stimulator may stimulate one or more afferent or efferent nerve fibers connected to the olfactory bulb, the pterygopalatine ganglia, and/or the pharyngeal branch of the vagus nerve.

In one or more embodiments, the nasal stimulator may be connected to one or more gas supply sources, such as, for example, a source of room air, medical air, oxygen, and/or a gas mixture including oxygen and one or more other gases (e.g., nitrogen, argon, carbon dioxide, helium, etc.). Other gases, such as, for example, medical gases (e.g., nitrous oxide), pharmaceuticals (e.g. albuterol, etc.), and/or anesthesia can also be introduced into the gas supply. The nasal stimulator may be connected to a gas-cylinder, a compressed gas-line, and/or an ambient source. In some embodiments, the nasal stimulator may draw air from the surrounding environment (or other sources), and process, clean, filter, humidify, heat, and/or cool the air. The nasal stimulator may adjust the pressure and flow rate of the gas source as required for therapeutic use.

For example, the nasal stimulator may be configured to nasally deliver gas at a flow rate of approximately 5 liters per minute (L/min) to approximately 70 L/min, depending on the needs of the patient, such as, for example, approximately 30 L/min to approximately 50 L/min. The flow rates of gas delivered by the nasal stimulator may be constant or may be varied. For example, the flow rate may be modulated in synchrony with a respiratory cycle of a patient, such as, for example, a respiratory cycle that includes an inspiration phase which has a duration of approximately 1.0 second to approximately 3.0 seconds, and an expiratory phase which has a duration of approximately 3.0 seconds to approximately 5.0 seconds. Delivery of gas via the nasal stimulator may be at a higher flow rate during an inspiration phase as compared to the flow rate of gas delivered during an expiration phase. Gas may be delivered via the nasal stimulator such that transnasal pressures are approximately 40 pascal (Pa) to approximately 80 Pa, or even less than approximately 40 Pa.

The nasal stimulator may include one or more sensors configured to receive data corresponding to a nerve activity, such as, for example, a nerve activity of a posterior superior alveolar nerve, a pterygopalatine ganglia, and/or a maxillary nerve. In addition or alternatively, one or more sensors of the nasal stimulator may be configured to receive data corresponding to a temperature, a pH, or a tissue wall tonus of a nasal cavity.

The nasal stimulator may include one or more electrodes. In some embodiments, each of the one or more electrodes may be configured to receive signals (e.g., act as a sensor receiving data corresponding to a nerve activity, a temperature, a pH, and/or a tissue wall tonus) and deliver signals (e.g., stimulation to one or more anatomical targets of the subject.) For example, when in place within a nasal canal of the subject, one or more electrodes of the nasal stimulator may be positioned to deliver stimulation to a maxillary nerve, a pterygopalatine ganglia, a posterior superior alveolar nerve, an afferent nerve fiber of a posterior superior alveolar nerve, and/or an efferent nerve fiber of a posterior superior alveolar nerve.

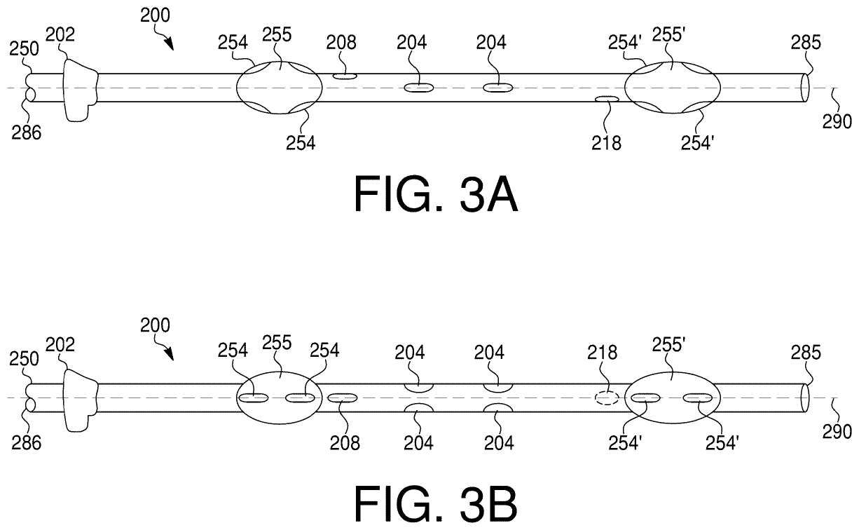

One exemplary configuration of a nasal stimulator 200 is shown in FIGS. 3A and 3B. The nasal stimulator 200 shown in FIG. 3B is the same nasal stimulator 200 shown in FIG. 3A, but the entire structure is rotated 90° about a longitudinal axis 290 that extends through the center of a lumen defined by the nasal stimulator 200. Nasal stimulator 200 may include a distal end 285 and a proximal end 286 opposite the distal end. The proximal end may be joined to a gas hose 250 via, for example nasal cavity interface 202. The gas hose 250 may extend and connect to a gas source, via, for example, a gas luer.

The nasal cavity interface 202 may form an air-tight seal with the nasal passages of the subject. In some embodiments, the nasal cavity interface 202 may form a seal with the nasal passages of the subject that is not air-tight (e.g., may allow gas to escape the nasal passages). For example, nasal cavity interface 202 may limit pressure levels by allowing gas to escape the nasal passage.

Nasal cavity interface 202 may form an interface between one or more lumens within nasal stimulator 200 and a gas hose 250. For example, gas may pass from a gas source (not pictured), through gas hose 250, to nasal cavity interface 202, through one or more lumens, and out one or more outflow ports 208 in a side wall of nasal stimulator 200. The outflow ports 208 may be configured to deliver gas to one or more anatomical targets (e.g., an olfactory bulb, a pterygopalatine ganglia, and/or a pharyngeal branch of a vagus nerve), as described herein. Electrical leads connecting nasal electrodes 204 to an energy source and/or a controller may pass through nose cavity interface 202 and/or gas hose 250 to the energy source and/or control unit.

The distal end 285 may be closed (e.g., forming a rounded tip; closing one or more lumens defined within nasal stimulator 200) or open (e.g., so that a lumen defined within nasal stimulator 200 is in fluid communication with the nasal passage, through the distal end 285). A nasal stimulator 200 including an open distal end 285 may be configured to deliver gas to a subject via the open distal end 285.

In some embodiments, the nasal stimulator 200 may include one or more occlusion devices. The one or more occlusion devices may be actuatable (e.g., inflatable). One or more occlusion devices may be configured to prevent stimulation from the nasal stimulator 200 (e.g., gas flow) from entering the lungs of the subject.

Still referring to FIGS. 3A and 3B, a nasal stimulator 200 may include a nasal cavity interface 202, one or more occlusion devices 255, one or more nasal electrodes 204, and one or more gas outlets 208 and/or gas inlets 218. In some embodiments, a nasal stimulator 200 may include two occlusion devices (e.g, occlusion device 255 and 255′). In some embodiments, multiple occlusion devices 255, 255′ may be inflated and/or deflated in combination with each of the other occlusion devices 255, 255′. In addition or alternatively, each occlusion device 255, 255′ may be inflated and/or deflated independently of one or more other occlusion devices 255, 255′. In embodiments, where multiple occlusion devices 255, 255′ are configured to be independently adjusted, nasal stimulator 200 may include multiple lumens for delivery of fluid (e.g., saline, air, etc.) to inflate the occlusion devices 255, 255′.

When occlusion device 255 and occlusion devices 255′ are both inflated, a length of a nasal passage may be closed off (e.g., sealed) between occlusion devices 255, 255′. Gas (e.g., gas for stimulation of an anatomical target) may be passed between gas outlet 208 and gas inlet 218 without entering other parts of the passage downstream or upstream of the bounded portion of the nasal passage, reducing the requisite pressure needed for stimulation of one or more anatomical targets. In some embodiments, gas may flow from the gas source, through gas hose 250, through nasal interface 202, through a first lumen of nasal stimulator 200, through a gas outlet 208 into a nasal cavity, through a gas inlet 218, through a second lumen of nasal stimulator 200, and out of distal end 285.

Each occlusion device 255, 255′ may further include one or more occlusion device electrodes 254, 254′. Placement of electrodes 254, 254′ on an occlusion device 255, 255′ may allow for electrodes 254, 254′ to be closer to tissue (e.g., closer to anatomical targets), as compared to electrodes 204. Similar to nasal electrodes 204, occlusion device electrodes 254, 254′ may be located at different radial positions about axis 290 of nasal stimulator 200. For example, two or more occlusion device electrodes 254, 254′ of each occlusion device 255, 255′ may be arranged in rows/lines (e.g., lines at different radial positions). For example, an occlusion device 255 may include at least two occlusion device electrodes 254 aligned along a longitudinal axis of the occlusion device 255. Lines of longitudinally aligned occlusion device electrodes 254 may be spaced at different radial positions of occlusion device 255 (e.g., two lines spaced 180° part, three lines spaced 120° apart, or four lines spaced 90° apart). One or more occlusion device electrodes 254 of one occlusion device 255, may be aligned or offset from one or more occlusion device electrodes 254′ of another occlusion device 255′.

Placement of one or more gas outlets 208 and/or gas inlets 218 between occlusion devices 255, 255′ may reduce the requisite gas pressure needed to stimulate one or more anatomical targets proximate the nasal canal. The placement of gas outlet 208 and gas inlet 218 in FIGS. 3A and 3B is exemplary, for example, the positions of gas outlet 208 and gas inlet 218 may be interchanged. In some embodiments, gas outlet 208 is radially spaced 180° apart from gas inlet 218, about axis 290. The gas flow from gas outlet 208 to gas inlet 218 may be coordinated with the gas flow from external respiratory support, and/or the subject's innate breath cycle, to enhance to the effectiveness of therapy.

Nasal stimulator 200 may include a securement means. The securement means may function either on the outside of the subject (e.g., straps wrapped around the subject's head) or inside the subject. The securement means may hold the nasal stimulator 200 in a fixed position, relative to the subject. The securement means may allow for one or more electrodes to be affixed in contact with the inner nose and/or exterior of the subject. In some embodiments, the one or more occlusion devices 255 may function as a securement means, when in an expanded configuration.

As described above, nasal stimulator 200 may include one or more lumens defined therewithin. For example, one or more lumens may provide for gas flow from the gas source, through nasal stimulator 200 to one or more gas outlets 208 (e.g., distal end 285). Further, the means for inflating one or more occlusion devices 255, 255′ (e.g., saline, air, or another fluid) may be provided from the source (e.g., gas source), through one or more lumens of nasal stimulator 200, to the occlusion device 255, 255′. In some embodiments, the electrical leads for occlusion device electrodes 254, 254′ and/or nasal electrodes 204, may be provided within one or more lumens of nasal stimulator 200. The electrical leads may be passed through one or more lumens containing gas or another fluid, or may be included in one or more separate lumens. The electrical leads may include wires, insulated metal leads, or metal (e.g., printed metal) embedded on and/or within one or more insulative materials.

Additional examples of nasal stimulators that may be used with embodiments of the present disclosure are described in WIPO Pub. No. WO 2021/144704, which is incorporated by reference herein.

In some embodiments, the stimulation system may include an oral endoscope. Examples of oral endoscopes that may be used with embodiments of the present disclosure are described in U.S. Pat. No. 10,940,308, which is incorporated by reference herein.

In some embodiments, the stimulation system may include one or more transvascular catheters. Examples of transvascular catheters that may be used with embodiments of the present disclosure are described in U.S. Pat. Nos. 9,242,088, 10,293,164, 10,039,920, 11,369,787, U.S. Pat. Pub. No. 2019/0001126, and U.S. Pat. Pub. No. 2020/0391027, each of which is incorporated by reference herein.

### Dermal Patch

In some embodiments, the stimulation system may further include one or more dermal patches that are configured to be affixed to the subject's skin. The dermal patch may be affixed to the skin of a subject via an adhesive or other means. A dermal patch may include one or more electrodes, such as, for example, electrodes configured to deliver electrical or magnetic stimulation. The electrodes of the dermal patch may be arranged in an array on or within the dermal patch, for example the electrodes may be arranged in a series of rows, a grid, and/or another shape that allows for placement of one or more electrodes proximate an anatomical target.

A dermal patch may be ovular, square, rectangular, elliptical, circular, triangular, or other suitable shape that allows for electrodes of the dermal patch to be arranged in a configuration proximate to one or more anatomical targets. A dermal patch may be flexible and able to conform to contours of the subject. In some embodiments, a dermal patch may be resilient and resistant to deformation.

A dermal patch may include a sensor configured to receive data related to a skin temperature, a skin pH, a tissue tonus (e.g., a gastric tonus, an intestinal tonus, and/or a muscle tonus), and/or electrical activity (e.g., EEG waves) from a nervous system of a subject. In some embodiments, one or more of the electrodes of the dermal patch may function as a sensor. The stimulation system may include one or more dermal patches, such as for example, one or more cranial dermal patches, one or more thoracic dermal patches, and/or one or more abdominal dermal patches.

Electrodes of the stimulation system, such as, for example, left lateral electrodes 350, right lateral electrodes 360, left medial electrodes 351, right medial electrodes 361, aural electrodes 450, nasal electrodes 204, occlusion device electrodes 254, dermal patch electrodes, and/or cranial electrodes, may comprise gold, copper, silver, platinum, graphite, graphene, another biologically compatible conductive material, or a combination thereof. In some embodiments, each electrode of the stimulation system has the same material composition. In other embodiments, different types of electrodes may have different material compositions. Electrodes may have a circular shape, a square shape, a triangular shape, a rectangular shape, or other two-dimensional shape.

Electrodes may have a length of approximately 1 mm to approximately 10 mm. Electrodes may have a width of approximately 1 mm to approximately 10 mm. For example, electrodes of oral stimulator 300 may have a width of approximately 1 mm to approximately 5 mm, and/or each electrode of oral stimulator 300 may have total area of approximately 1 square millimeter (mm²) to approximately 50 mm². Electrodes of nasal stimulator 200 may have a width of approximately 1 mm to approximately 10 mm, and/or each electrode of nasal stimulator 200 may have total area of approximately 1 mm² to approximately 100mm². Electrodes of aural stimulator 400 may have a width of approximately 1 mm to approximately 3 mm, and/or each electrode of aural stimulator 400 may have total area of approximately 1 square mm² to approximately 30 mm².

A stimulation system may include, be in communication with, or be configured to communicate with one or more external respiratory support devices. Exemplary external respiratory support devices include mechanical ventilators, CPAP machines, and/or high-low flow oxygen masks.

### Control Unit

A stimulation system may include a control unit configured to receive data from one or more sensors, process data from one or more sensors, coordinate the delivery of stimulation from one or more components of the stimulation system, coordinate delivery of external respiratory support from an external respiratory device, and/or adjust stimulation parameters of one or more channels of stimulation. Components of the stimulation system (e.g., oral stimulators, aural stimulators, nasal stimulators, oral endoscopes, transvascular catheters, dermal patches, and/or external respiratory support devices) may be in communication with each other and/or the control unit. For example, components of the stimulation system may communicate via a wired or wireless (e.g., Wi-Fi, RF, Bluetooth) connection.

The control unit may control the supply of stimulation energy (e.g., gas flow, electrical current, infrasonic waves) to oral stimulators, aural stimulators, nasal stimulators, oral endoscopes, transvascular catheters, dermal patches, and/or external respiratory support devices. Further, the controller may communicate with one or more sensors. Data collected from the one or more sensors may be used to adjust one or more stimulation parameters. Stimulation parameters may include a duration, a pulse width, a frequency, an amplitude, or a combination thereof.

This adjustment of stimulation parameters may be performed by the control unit, another unit or system, or a user. In some embodiments, data may be received/exchanged with another device (e.g., a diagnostic device, a therapeutic device, etc.). The other device may be in communication with the patient. In some embodiments, data may be exchanged with an external respiratory support system and/or one or more sensors connected to an external respiratory support system.

The control unit may manage the delivery of stimulation, such as, for example, mechanical stimulation via gas flow, electrical stimulation, magnetic stimulation, mechanical stimulation via intranasal cavity pressure modulation, thermal stimulation, infrared stimulation, electromagnetic stimulation, infrasonic stimulation, or a combination thereof. Stimulation may be delivered, as coordinated by the control unit, from multiple sources, such as, for example, electrical stimulation from multiple electrodes, gas flow from multiple gas flow sources, infrared stimulation from multiple infrared input and output sources, and/or electromagnetic stimulation from a multi-dimensional electromagnetic field. The control unit may coordinate information between one or more sensors, energy sources, other components of the system, and one or more external respiratory support devices. The controller may also interface with one or more external respiratory support devices, such as, for example, a mechanical ventilator, to control delivery of external respiratory support or positive pressure gas.

The control unit may be configured to deliver stimulation energy in synchronization with a breath cycle, such as, for example, a breath cycle of an external respiratory support device and/or a subject's innate breath cycle. In some embodiments, one or more anatomical targets may be stimulated in synchrony with the breathing cycle of a subject, such as for example, an innate breathing cycle or a breathing cycle regulated by one or more external respiratory support systems.

Stimulating anatomical targets (e.g., a phrenic nerve, a vagus nerve, a pterygopalatine ganglia, a celiac ganglion, a superior cervical ganglia, a great petrosal nerve, a maxillary nerve, a posterior superior alveolar nerve, an afferent nerve fiber of a posterior superior alveolar nerve, and/or an efferent nerve fiber of a posterior superior alveolar nerve) in coordination with the breathing cycle of a subject may modulate one or more brain networks and/or improve glymphatic system flow. Further, stimulating anatomical targets in coordination with the breathing cycle may provide beneficial physiologic responses related to the promotion of healthy cerebral autoregulation, such as, for example, modulating myogenic activity in cerebral blood vessels, activating smooth muscles in cerebral blood vessels, or both.

Additional features and aspects of control units that may be used with embodiments of the present disclosure are described in U.S. Pat. Nos. 9,333,363, 9,776,005, 10,940,308, 10,987,511, 11,357,979, U.S. Pat. Pub. No. 2019/0001126, U.S. Pat. Pub. No. 2020/0391027, U.S. Pat. Pub. No. 2022/0134095, and WIPO Pub. No. 2021/144704, each of which is incorporated by reference herein.

A stimulation system may include one or more sensors configured to measure a physiological property of the subject, a characteristic of applied therapy, or both. The one or more sensors may measure one or more parameters of energy delivery (e.g., stimulation energy delivered), an electrical activity and/or potential representative of nerve or muscle activity, a distance between two sources of infrared energy, a flowrate of, for example, a delivered gas, one or more diameters of one or more occlusion devices, an absorbance, a transmittance, a reflectance, an impedance, a magnetic field direction, a magnetic field magnitude, a pressure, or a combination thereof.

In some embodiments, the stimulation system may receive data from one or more sensors, and control or modulate applied stimulation based on the data received from the one or more sensors (e.g., a closed-loop system). For example, the control unit may be configured to receive data from one or more sensors. The sensors may be electrically coupled to energy sources and/or configured to include a battery. Sensors and other components of the system can communicate with each other via wired or non-wired connections (e.g., Wi-Fi, RF, etc.).

In some embodiments, the data received by the one or more sensors may provide information regarding physiological responses of the subject to stimulation. Data received from the one or more sensors may include EEG waves, electrical impulses indicative of nerve activity, absorbance, cerebral myogenic frequency, pupil diameter, distance between optic nerve and optic nerve shaft, retinal vessel diameter, heart rate, heart rate variability, skin temperature, temperature and/or pH of one or more anatomical lumens (e.g., a blood vessel including a transvascular catheter, an esophagus, a nasal cavity, a mouth), tissue tonus, muscle tonus, intestinal tonus, gastric tonus, and/or gastric pH.

The one or more sensors may provide data to the stimulation system (e.g., real-time feedback) regarding one or more physiological parameters of the subject. For example, the one or more sensors may transmit data (e.g., wirelessly) to a control unit of the stimulation system. The one or more physiological parameters of the subject may be indicative of the subject's response to stimulation delivered via a stimulation system comprising a dermal patch, a nasal stimulator, an oral stimulator, an aural stimulator, an oral endoscope, and/or a transvascular catheter.

In some embodiments, the stimulation system may include one or more sensors disposed on or within a nasal stimulator, oral stimulator, aural stimulator, oral endoscope, and/or transvascular catheter. In addition or alternatively, the stimulation system may be in communication with one or more external sensors such as, for example, cranial dermal patches, ocular sensors, thoracic dermal patches, and/or abdominal dermal patches.

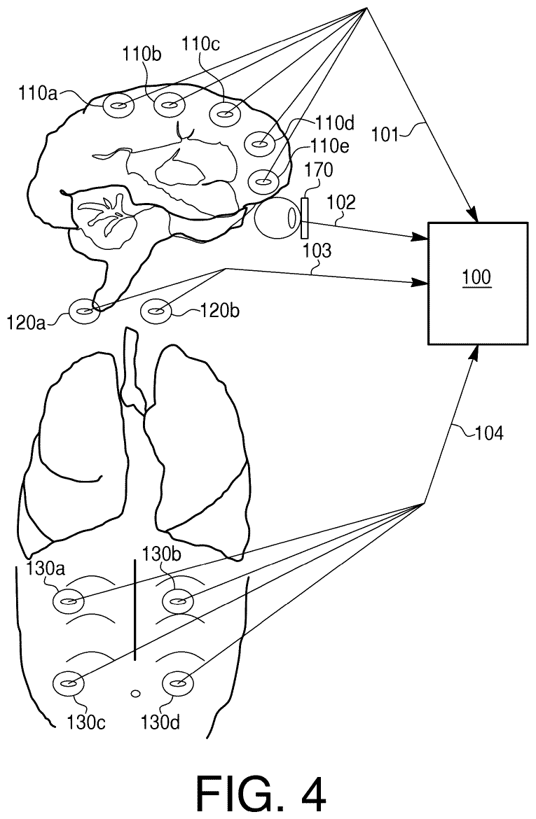

Referring to FIG. 4, the control unit 100 may be in communication with one or more external sensors, including cranial dermal patches 110, ocular sensors 170, thoracic dermal patches 120, and/or abdominal dermal patches 130. The external sensors may provide data regarding physiological parameters of the subject to the control unit 100.

For example, one or more cranial dermal patches 110 may be placed on the cranium of the subject. In one or more embodiments, the one or more cranial dermal patches 110 include a first cranial dermal patch 110a placed on a prefrontal cortex, a second cranial dermal patch 110b placed on a respiratory afferent cortex, a third cranial dermal patch 110c placed on a respiratory efferent cortex, a fourth cranial dermal patch 110d placed on a parietal cortex, and a fifth cranial dermal patch 110e placed on a temporal cortex. Each of the cranial dermal patches 110 may include a sensor configured to measure brain electrical activity.

For example, the cranial dermal patches 110 may include one or more electrodes configured to record the electrical activity of one or more regions of the subject's brain, over a period of time. The cranial dermal patches 110, or a system in communication with cranial dermal patches 110 (e.g., control unit 100) may generate an electroencephalogram (EEG) based on the recorded electrical activity of the brain. In some embodiments, a cranial dermal patch 110 may include an infrared optode configured to measure a cerebral blood flow, a cerebral vasodilation, and/or a cerebral myogenic frequency.

The one or more cranial dermal patches 110a, 110b, 110c, 110d, 110e may transmit cranial data 101 to control unit 100. The cranial data 101 may include data related to EEG waves, cerebral blood flow, cerebral vasodilation, cerebral myogenic frequency, and/or skin temperature.

One or more ocular sensors 170 may be placed proximate an eye of the subject. An ocular sensor 170 may be configured to measure a pupil diameter, a distance between an optic nerve and an optic nerve shaft, and/or a retinal vessel diameter. One or more ocular sensors 170 may transmit ocular data 102 to control unit 100. The ocular data 102 may include data related to a pupil diameter, a distance between an optic nerve and an optic nerve shaft, and/or retinal vessel diameter.

One or more thoracic dermal patches 120a, 120b may be placed on a thorax of the subject. For example, a first thoracic dermal patch 120a and a second thoracic dermal patch 120b may be placed on the first intercostal space, tenth intercostal space, and/or between the first and tenth intercostal spaces. The first thoracic dermal patch 120a and second thoracic dermal patch 120b may be placed on an anterior middle line, a posterior middle line, an anterior lateral line, a posterior lateral line, or a combination thereof.

A thoracic dermal patch 120 may be configured to measure a skin temperature, a tissue tonus (e.g., a muscle tonus), a heart rate, and/or a heart rate variability. One or more thoracic dermal patches 120a, 120b may transmit thoracic data 103 to control unit 100. Thoracic data 103 may include data related to a skin temperature, a tissue tonus (e.g., a muscle tonus), a heart rate, and/or a heart rate variability.

One or more abdominal dermal patches 130a, 130b, 130c, 130d may be placed on an abdomen of the subject. For example, a first abdominal dermal patch 130a may be placed on a left abdominal lateral line, a second abdominal dermal patch 130b may be placed the left abdominal lateral line, a third abdominal dermal patch 130c may be placed on a right abdominal lateral line, and a fourth abdominal dermal patch 130d may be placed the right abdominal lateral line.

An abdominal dermal patch 130 may be configured to measure a skin temperature, a tissue tonus (e.g., a muscle tonus, a gastric tonus, an intestinal tonus), a heart rate, and/or a heart rate variability. One or more abdominal dermal patches 130a, 130b, 130c, 130d may transmit abdominal data 104 to control unit 100. Abdominal data 104 may include data related to a skin temperature, a tissue tonus (e.g., a muscle tonus, a gastric tonus, an intestinal tonus), a heart rate, and/or a heart rate variability.

One or more sensors may include an accelerometer configured to determine timing of one or more components of a breath cycle (e.g., inspiration duration, inspiration pause, expiration duration, and/or expiration pause). For example, an accelerometer may be incorporated into one or more sensors of on or within an oral stimulator, an aural stimulator, a nasal stimulator, an oral endoscope, and/or a transvenous catheter.

Control unit 100 may adjust one or more stimulation parameters of stimulation delivered by one or more components of the stimulation system based on cranial data 101, ocular data 102, thoracic data 103, and/or abdominal data 104. In addition or alternatively, control unit 100 may adjust one or more stimulation parameters based on data received from one or more sensors on or within an oral stimulator, an aural stimulator, a nasal stimulator, an oral endoscope, and/or a transvenous catheter.

In some embodiments, control unit 100 may adjust one or more stimulation parameters until physiological parameters of the subject indicate a positive response to stimulation delivered by one or more components of the stimulation system. For example, control unit 100 may adjust one or more stimulation parameters if there is not an abdominal skin temperature increase of at least approximately 0.5-1.0 Celsius degrees. Control unit 100 may adjust one or more stimulation parameters if there is not at least approximately a 5% decrease in abdominal muscle tonus. Control unit 100 may adjust one or more stimulation parameters if there is not an observed increase in heart rate variability. Control unit 100 may adjust one or more stimulation parameters if there is not an observed pupil diameter increase of at least approximately 10%. Control unit 100 may adjust one or more stimulation parameters if there is not an increase in alpha and theta EEG waves. Control unit 100 may adjust one or more stimulation parameters if there is not a decrease in overall frequency power in the prefrontal cortex. Control unit 100 may adjust one or more stimulation parameters if a reduction in cerebral myogenic frequency is not observed.

As described herein, energy (e.g., electrical or magnetic) may be passed between two or more electrodes (e.g., left lateral electrodes 350, right lateral electrodes 360, left medial electrodes 351, right medial electrodes 361, aural electrodes 450, nasal electrodes 204, occlusion device electrodes 254, dermal patch electrodes, and/or cranial electrodes) to provide stimulation to one or more anatomical targets, such as, for example, a phrenic nerve, a vagus nerve, a pterygopalatine ganglia, a celiac ganglion, a superior cervical ganglia, a great petrosal nerve, a maxillary nerve, a posterior superior alveolar nerve, an afferent nerve fiber of a posterior superior alveolar nerve, and/or an efferent nerve fiber of a posterior superior alveolar nerve.

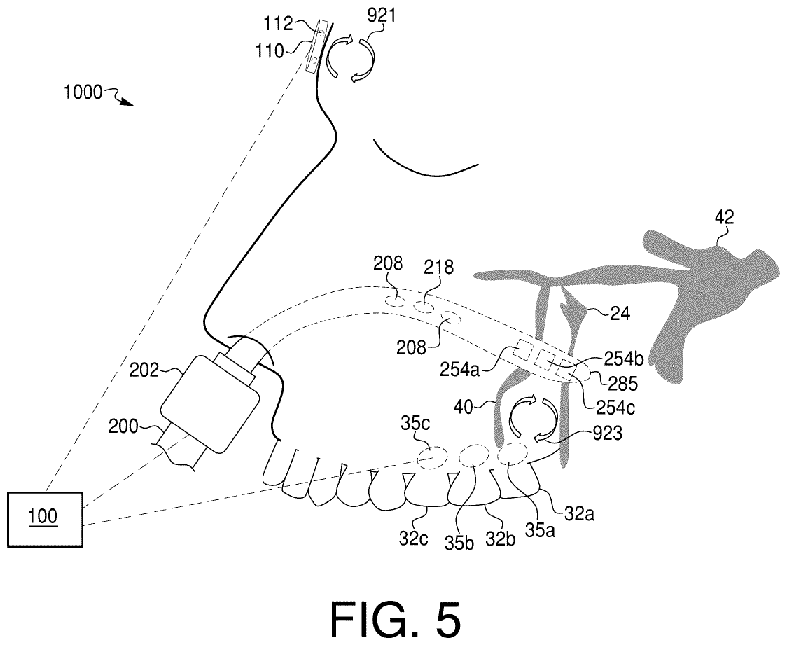

Referring to FIG. 5, a stimulation system 1000 may include a nasal stimulator 200, a cranial dermal patch 110, and a control unit 100. The stimulation system 1000 shown in FIG. 5 may also include an oral stimulator 300, however, the oral stimulator 300 is not shown in FIG. 5 in order to provide a clearer view of left first molar 32a, left second molar 32b, left third molar 32c, and oral stimulation targets 35a, 35b, 35c. In some embodiments, oral stimulator 300 may be configured to stimulate anatomical targets above a subject's upper teeth, below a subject's lower teeth, or both. Stimulation system 1000 may include a first oral stimulator 300 configured to stimulate anatomical targets above a subject's upper teeth, and a second oral stimulator 300 configured to stimulate anatomical targets below the subject's lower teeth.

Oral stimulation targets 35a, 35b, 35c may include tissue above the left molars of the subjects, such as, for example, gum tissue, nerve tissue, and muscle tissue. The nerve tissue of oral stimulation targets 35a, 35b, 35c may include a posterior superior alveolar nerve 40, nerve roots, afferent nerve fibers connected to the posterior superior alveolar nerve 40, efferent nerve fibers connected to the posterior superior alveolar nerve 40, a maxillary nerve 42, a pterygopalatine ganglia 24, a superior cervical ganglia, and/or a greater petrosal nerve.

In some embodiments, oral stimulator 300 may be placed such that oral stimulation target 35a is between first left lateral electrode 350a and first left medial electrode 351a, oral stimulation target 35b is between second left lateral electrode 350b and second left medial electrode 351b, and/or oral stimulation target 35c is between third left lateral electrode 350c and third left medial electrode 351c.

Although not shown in FIG. 5, stimulation system 1000 may be configured to stimulate one or more anatomical targets on the right side of a subject's head (e.g., one or more anatomical targets proximate to a right first molar, a right second molar, and/or a right third molar). For example, oral stimulator 300 may be placed such that a first oral stimulation target is between first right lateral electrode 360a and first right medial electrode 361a, a second oral stimulation target is between second right lateral electrode 360b and second right medial electrode 361b, and/or a third oral stimulation target is between third right lateral electrode 360c and third left medial electrode 361c.

Referring again to FIG. 5, cranial dermal patch 110 may include one or more cranial electrodes 112. Although two cranial electrodes 112 are shown in FIG. 5, cranial dermal patch 110 may have any suitable number of electrodes. Dermal patch 110 may include one or more sensors configured to receive data corresponding to cortical brain wave activity and/or optical amplitude modulation. In some embodiments, one or more cranial electrodes 112 may function as a sensor. Dermal patch 110 may receive signals (e.g., electroencephalogram (EEG) waves 921) from one or more of the prefrontal cortex, the sensory cortex, the motor cortex, the parietal cortex, and the temporal cortex. In some embodiments, stimulation system 1000 includes a plurality of cranial dermal patches 110 configured to receive signals from one or more of the prefrontal cortex, the sensory cortex, the motor cortex, the parietal cortex, and the temporal cortex.

In some embodiments, cranial dermal patch 110 may be configured to deliver a stimulation signal (e.g., via one or more cranial electrodes 112). Stimulation signals from dermal patch 110 may be delivered to a prefrontal cortex, a supplementary motor cortex, a diaphragm motor cortex, or a combination thereof.

Nasal stimulator 200 may include a nasal interface 202. Nasal stimulator 200 may also include one or more gas inlets 218, and or one or more gas outlets 208. Gas may be transferred from the gas source, through nasal interface 202, to the one or more gas outlets 208. While nasal stimulator is within a nasal canal of the subject, the one or more gas outlets 208 may be configured to provide gas flow that stimulates an olfactory bulb, a pterygopalatine ganglia, and/or a pharyngeal branch of a vagus nerve.

In some embodiments, nasal stimulator 200 may include one or more nasal electrodes 254. For example, the nasal electrodes 254 may be closer to a distal end of nasal stimulator 200 than the gas inlets 218 and gas outlets 208. The embodiment shown in FIG. 5 includes at least three nasal electrodes 254a, 254b, 254c.

One or more nasal electrodes 254a, 254b, 254c may function as a sensor and receive data regarding nerve activity. For example, the one or more nasal electrodes 254a, 254b, 254c may receive electrical impulses 923 from one or more nerves proximate to the nasal electrodes 254a, 254b, 254c. In addition or alternatively, one or more nasal electrodes 254a, 254b, 254c may receive data regarding a temperature, a pH, and/or a tissue wall tonus of a nasal cavity.

During therapy, stimulation may be delivered from one or more of the nasal electrodes 254a, 254b, 254c to one or more anatomical targets, such as, for example, a posterior superior alveolar nerve, afferent nerve fibers connected to the posterior superior alveolar nerve, efferent nerve fibers connected to the posterior superior alveolar nerve 40, a maxillary nerve 42, a pterygopalatine ganglia 24, a superior cervical ganglia, and/or a greater petrosal nerve.

In some embodiments, stimulation may be delivered simultaneously, or sequentially, from oral stimulator 300 and nasal stimulator 200. The delivery of stimulation from oral stimulator 300 and nasal stimulator 200 may generate a multi-dimensional electromagnetic field between electrodes of the oral stimulator and nasal electrodes 254. The multi-dimensional electromagnetic field may stimulate a posterior superior alveolar nerve 40, efferent nerve fibers connected to the posterior superior alveolar nerve 40, afferent nerve fibers connected to the posterior superior alveolar nerve 40, a maxillary nerve 42, a pterygopalatine ganglia 24, a superior cervical ganglia, and/or a greater petrosal nerve.

Stimulation system 1000 may include a control unit 100. As described herein, control unit 100 may be in communication (e.g., wireless communication) with one or more components of stimulation system 1000. Control unit 100 may receive information from cranial dermal patch 110, nasal stimulator 200, and/or oral stimulator 300. For example, control unit 100 may receive data from one or more sensors regarding EEG waves 921, electrical impulses 923, temperature, tissue tonus, and/or pH. Control unit 100 may coordinate the delivery and stimulation parameters of stimulation delivered from dermal patch 110, nasal stimulator 200, and/or oral stimulator 300.

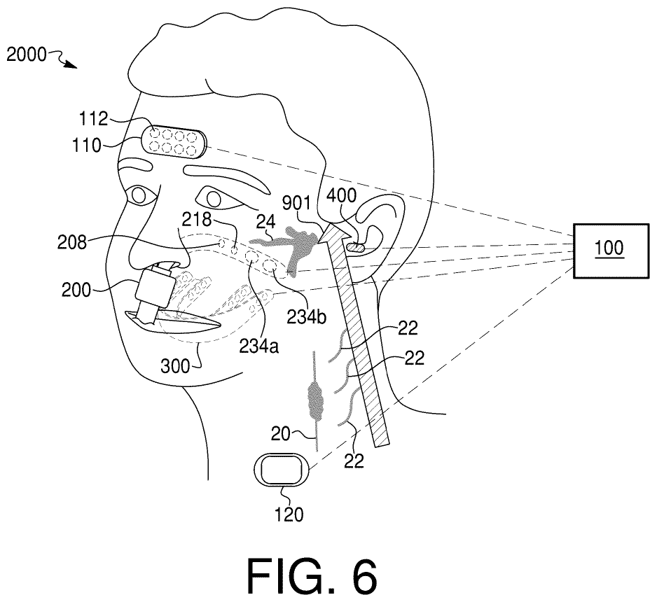

Referring to FIG. 6, a stimulation system 2000 may include one or more cranial dermal patches 110, one or more thoracic dermal patches 120, a nasal stimulator 200, an oral stimulator 300, one or more aural stimulators 400, and/or a control unit 100.

A thoracic dermal patch 120 may include one or more sensors configured to receive information regarding a skin temperature or muscle tonus of the subject. Although not shown in FIG. 6, stimulation system 2000 may also include an oral endoscope and/or a transvascular catheter. The oral endoscope and/or transvascular catheter may include one or more electrodes configured to stimulate a phrenic nerve. The stimulation delivered by the one or more components of stimulation system 2000 may cause retrograde nerve stimulation 901 of the superior cervical ganglia 20 and phrenic nerve branches 22 of the spinal cord.

In some embodiments, stimulation delivered by the stimulation system 2000 may generate a multi-dimensional electromagnetic field between two or more of the nasal stimulator 200, the oral stimulator 300, and the aural stimulator 400. The multi-dimensional electromagnetic field may stimulate a maxillary nerve 42, a pterygopalatine ganglia 24, a superior cervical ganglia, and/or a greater petrosal nerve.

Stimulation system 2000 may include a control unit 100. As described herein, control unit 100 may be in communication (e.g., wireless communication) with one or more components of stimulation system 2000. Control unit 100 may receive information from cranial dermal patch 110, thoracic dermal patch 120, nasal stimulator 200, oral stimulator 300, and/or aural stimulator 400. For example, control unit 100 may receive data from one or more sensors regarding EEG waves 921, airway pressure, internal pressure of an anatomical cavity (e.g., a nasal cavity, an ear canal, a vascular lumen), location of aural stimulator 400, membrane of tympanum tension, nasal cavity temperature, nasal cavity pH, oral cavity temperature, oral cavity pH, ear canal temperature, ear canal pH, skin temperature, and/or muscle tonus. Control unit 100 may coordinate the delivery and stimulation parameters of stimulation delivered from nasal stimulator 200, oral stimulator 300, and/or aural stimulator 400.

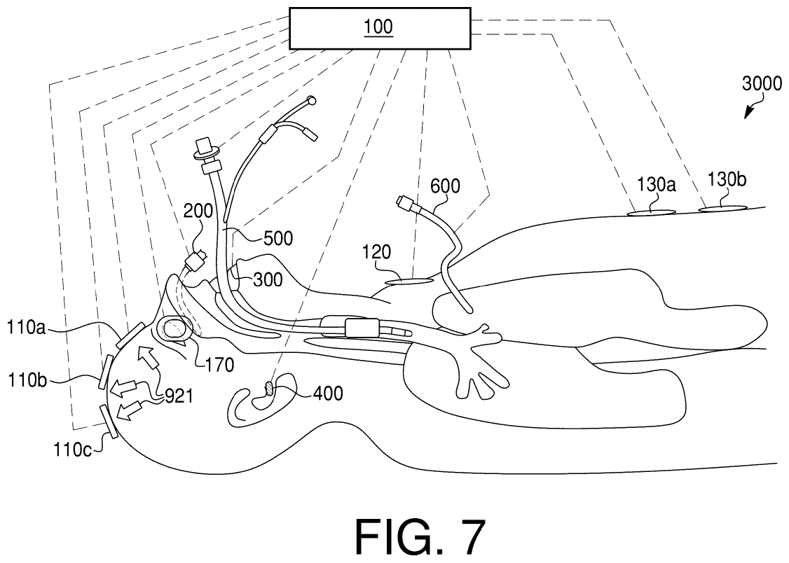

Referring to FIG. 7, a stimulation system 3000 may include one or more cranial dermal patches 110, one or more thoracic dermal patches 120, one or more abdominal dermal patches 130, an ocular sensor 170, a nasal stimulator 200, an oral stimulator 300, an oral endoscope 500, a transvascular catheter 600, and/or a control unit 100.

In some embodiments, stimulation system 3000 may include a first cranial dermal patch 110a, a second cranial dermal patch 110b, and a third cranial dermal patch 110c. Each cranial dermal patch 110 may be placed at a different location on the cranium of the subject. Each cranial dermal patch 110 may receive EEG waves 921 from one or more of the prefrontal cortex, the sensory cortex, the motor cortex, the parietal cortex, and the temporal cortex.

Stimulation system 3000 may include an ocular sensor 170 positioned proximate an eye of the subject. The ocular sensor 170 may be configured to measure a pupil diameter, a distance between an optic nerve and an optic nerve shaft, and/or a retinal vessel diameter. The ocular sensor 170 may transmit data relating to a pupil diameter, a distance between an optic nerve and an optic nerve shaft, and/or a retinal vessel diameter to control unit 100.

In some embodiments, stimulation system 3000 includes one or more abdominal dermal patches 130a, 130b. The abdominal dermal patches 130a, 130b may be configured to measure a skin temperature, a tissue tonus (e.g., a muscle tonus, a gastric tonus, an intestinal tonus), a heart rate, and/or a heart rate variability. Abdominal dermal patches 130a, 130b may transmit data related to a skin temperature, a tissue tonus (e.g., a muscle tonus, a gastric tonus, an intestinal tonus), a heart rate, and/or a heart rate variability to control unit 100.

As described herein, control unit 100 may be in communication (e.g., wireless communication) with one or more components of stimulation system 3000. Control unit 100 may receive information from the one or more cranial dermal patches 110, the one or more thoracic dermal patches 120, the one or more abdominal dermal patches 130, the ocular sensor 170, the nasal stimulator 200, the oral stimulator 300, the oral endoscope 500, and the transvascular catheter 600. For example, control unit 100 may receive data from one or more sensors regarding EEG waves 921, airway pressure, internal pressure of an anatomical cavity (e.g., a nasal cavity, an ear canal, a vascular lumen), location of aural stimulator 400, membrane of tympanum tension, nasal cavity temperature, nasal cavity pH, oral cavity temperature, oral cavity pH, ear canal temperature, ear canal pH, skin temperature, and/or muscle tonus. Control unit 100 may coordinate the delivery and stimulation parameters of stimulation delivered from nasal stimulator 200, oral stimulator 300, aural stimulator 400, oral endoscope 500, and/or transvascular catheter 600.

The different embodiments of the various stimulation system components may be combined and used together in any logical arrangement. Furthermore, individual features or elements of any described embodiment may be combined with or used in connection with the individual features or elements of other embodiments. It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification be considered as exemplary only.

1-8. (canceled)

9. A stimulation system comprising:
an oral stimulator including:
a front wall;
a rear wall;
a first electrode and a second electrode positioned on or within the front wall;
a third electrode and a fourth electrode positioned on or within the rear wall;
wherein the first and third electrodes are configured to stimulate one or more nerve fibers above a left molar; and
wherein the second and fourth electrodes are configured to stimulate one or more nerve fibers above a right molar.

10. The stimulation system of claim 9, further comprising a cranial dermal patch comprising one or more electrodes configured to receive EEG waves from a brain.

11. The stimulation system of claim 10, further comprising a control unit in communication with the cranial dermal patch and the oral stimulator;
wherein the control unit is configured to adjust stimulation delivered by the first, second, third, and fourth electrodes, based on EEG waves received from the cranial dermal patch.

12. The stimulation system of claim 9, wherein one or more of the first, second, third, or fourth electrodes are configured to receive an electrical impulse indicative of a nerve activity.

13. The stimulation system of claim 9, further comprising a nasal stimulator connected to a gas source, wherein the nasal stimulator comprises:
a gas outflow; and
a nasal electrode.

14. The stimulation system of claim 13, wherein the nasal electrode and one or more of the first, second, third, and fourth electrodes, are configured to generate an electromagnetic field between the nasal stimulator and the oral stimulator that stimulates a maxillary nerve, a pterygopalatine ganglia, a superior cervical ganglia, and/or a posterior superior alveolar nerve.

15. The stimulation system of claim 14, wherein the nasal stimulator and the oral stimulator are in communication with a control unit configured to coordinate the generation of the electromagnetic field with a breathing cycle of a subject.

16. A stimulation system comprising:
an aural stimulator including:
a first sensor configured to emit and receive sub-audible waves;
a second sensor configured to measure a pressure applied to the aural stimulator;
a control unit in communication with the aural stimulator;
wherein the aural stimulator is configured to transmit data regarding the emitted and received sub-audible waves to the control unit;
wherein the control unit is configured to receive the data regarding the emitted and received sub-audible waves from the aural stimulator, and based on the received data, determine a tension of tissue.

17. The stimulation system of claim 16, wherein the aural stimulator further includes a third sensor configured to measure a distance between a cranial wall and a ventral wall of an ear canal.

18. The stimulation system of claim 17, wherein the aural stimulator is configured to transmit data regarding the pressure applied to the aural stimulator and/or the distance between the cranial and ventral walls of the ear canal, to the control unit; and
the control unit is configured to receive data regarding the emitted and received sub-audible waves, the pressure applied to the aural stimulator, and/or the distance between the cranial and ventral walls of the ear canal, from the aural stimulator.

19. The stimulation system of claim 18, further comprising a transvascular catheter configured to deliver stimulation to a phrenic nerve, wherein the control unit is configured to adjust the stimulation delivered by the transvascular catheter, based on data received by the control unit from the aural stimulator.

20. The stimulation system of claim 18, further comprising a nasal stimulator, an oral stimulator, or both, wherein the nasal stimulator, oral stimulator, or both are configured to deliver stimulation to a maxillary nerve, a pterygopalatine ganglia, a superior cervical ganglia, and/or a posterior superior alveolar nerve; and
wherein the control unit is configured to adjust the stimulation delivered by the nasal stimulator, the oral stimulator, or both, based on data received by the control unit from the aural stimulator.

21. The stimulation system of claim 18, further comprising an oral endoscope configured to deliver stimulation to a phrenic nerve, a vagus nerve, or both; and
wherein the control unit is configured to adjust the stimulation delivered by the oral endoscope, based on data received by the control unit from the aural stimulator.

22. A stimulation system comprising:
an oral stimulator including:
a front wall;
a rear wall;
a first electrode positioned on or within the front wall;
a second electrode positioned on or within the rear wall;
wherein the first and second electrodes are configured to stimulate one or more nerve fibers above a molar; and
an aural stimulator including:
a first sensor configured to emit and receive sub-audible waves;
a second sensor configured to measure a pressure applied to the aural stimulator.

23. The stimulation system of claim 22, further comprising a cranial dermal patch including:
one or more electrodes configured to receive EEG waves from a brain; and
an infrared optode configured to measure a cerebral myogenic frequency.

24. The stimulation system of claim 22, further comprising a thoracic patch configured to receive data related to a first skin temperature, a first tissue tonus, and/or a heart rate variability.

25. The stimulation system of claim 22, further comprising an abdominal dermal patch configured to receive data related to a second skin temperature, a second tissue tonus, or both.

26. The stimulation system of claim 22, further comprising an ocular sensor configured to receive data related to a pupil diameter, a distance between an optic nerve and an optic nerve shaft, a retinal vessel diameter, or a combination thereof.

27. The stimulation system of claim 22, further comprising an external respiratory support device configured to provide respiratory assistance to a subject, and a control unit configured to receive data from the aural stimulator, the cranial dermal patch, a thoracic dermal patch, an abdominal dermal patch, and an ocular sensor, wherein the received data relates to:
the EEG waves from the brain;
the cerebral myogenic frequency;
the first skin temperature;
the second skin temperature;
the first tissue tonus;
the second tissue tonus;
the heart rate variability;
the pupil diameter;
the distance between the optic nerve and the optic nerve shaft;
the retinal vessel diameter; or
a combination thereof; and
wherein the control unit is configured to coordinate stimulation delivered by the oral stimulator with the respiratory assistance provided by the external respiratory support device.

28. The stimulation system of claim 27, wherein control unit is configured to modify the stimulation delivered by the oral stimulator based on the data received from the aural stimulator, the cranial dermal patch, the thoracic dermal patch, the abdominal dermal patch, and the ocular sensor.
